(19) United States
(12) Patent Application Publication
(10) Pub. No.: US 2004/0048773 A1
(43) Pub. Date: Mar. 11, 2004

(54) Use of leptin antagonists for the treatment of diabetes

(75) Inventors: Michael Cawthorne, Horsham (GB); Valur Emilsson, Kopavogur (IS); Yong-Ling Liu, Great Bookham (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham p.l.c.

(21) Appl. No.: 10/274,254

(22) Filed: Oct. 17, 2002

Related U.S. Application Data
(63) Continuation of application No. 10/133,285, filed on Apr. 26, 2002, now abandoned.
Application No. 10/133,285 is a continuation of application No. 09/445,524, filed on Mar. 24, 2000, now abandoned.
Application No. 09/445,524 is a 371 of international application No. PCT/EP97/02240, filed on Jun. 6, 1997.

(30) Foreign Application Priority Data
Apr. 26, 1996 (GB) 9608648.3

(51) Int. Cl. A61K 31/00

(52) U.S. Cl. 514/1

(57) ABSTRACT

The use of an antagonist of leptin for the manufacture of a medicament for the treamtne tof disorders resulting from deficiencies in insulin secretion, hyperglycaemia and insulin resistance.

The invention relates to a novel use, in particular a use for the treatment of diabetes and complications thereof.

Non-insulin-dependent diabetes (NIDDM) is known to be caused by insulin resistance (particularly in skeletal muscle, adipose tissue and liver) and an inadequate insulin secretion from the beta-cells of the Islets of Langerhans in the pancreas. Thus, despite hyperinsulinaemia there is insufficient insulin to compensate for the insulin resistance and to maintain blood glucose in the desirable range.

Pelleymounter et al (Science, 1995, 269, 540-543) have reported that the ob polypeptide or “leptin” lowers both plasma insulin and glucose levels in the genetically obese ob/ob mouse.

United Kingdom patent application, Publication Number 2292382 relates inter alia to polypeptides, OB polypeptides, and antagonists thereof and their use for modulating bodyweight. The disclosures of GB 2292382 are incorporated herein by reference.

We have now shown that recombinant leptin directly inhibits insulin release from both isolated islets and the perfused pancreas of the ob/ob mouse. An antagonist of leptin is therefore indicated to be of value in enhancing insulin secretion and thereby assisting in the control of blood glucose levels. We have further shown that leptin inhibits basal and insulin-stimulated glycogen synthesis in isolated soleus muscle of ob/ob mice. An antagonist of leptin is therefore also indicated to be of value in enhancing glucose utilisation and the action of insulin to enhance glucose utilisation. Antagonists are therefore indicated to be of direct use in the treatment of disorders resulting from deficiencies in insulin secretion and action and of hyperglycaemia, such as non-insulin-dependent diabetes. Moreover, since hyperglycaemia is believed to lead to many of the long term complications of diabetes, an antagonist which enhances insulin secretion and assists in blood glucose control, may be useful in the treatment of diabetic complications, such as retinopathy, nephropathy and angiopathy.

Accordingly, the invention provides the use of an antagonist of leptin for the treatment of disorders resulting from deficiencies in insulin secretion and of hyperglycaemia, such as non-insulin-dependent diabetes (NIDDM).

In a further aspect, there is provided the use of an antagonist of leptin for the manunfacture of a medicament for the treatment of disorders resulting from deficiencies in insulin secretion and of hyperglycaemia, such as non-insulin-dependent diabetes (NIDDM).

Suitable antagonists of leptin are as disclosed in GB2292382 and may be prepared according to methods disclosed therein.

Particular antagonists include protein antagonists.

Particular antagonists include non-protein antagonists, especially small organic molecule antagonists.

The present invention also extends to a method for the treatment of disorders resulting from deficiencies in insulin secretion and of hyperglycaemia, such as non-insulin-dependent diabetes (NIDDM), in a human or non-human mammal, which method comprises the administration to human or non-human mammal in need of such treatment, an effective, pharmaceutically acceptable, non-toxic amount of an antagonist of leptin.

The present invention also extends to a pharmaceutical composition comprising an antagonist of leptin, or a pharmaceutically acceptable derivative thereof, and a pharmaceutically acceptable carrier.

Particular compositions of the invention are those used for the treatment of disorders resulting from deficiencies in insulin secretion and of hyperglycaemia, such as non-insulin-dependent diabetes (NIDDM).

Suitable pharmaceutically acceptable carriers are as dictated by conventional practice such as those disclosed in GB2292382 or in International Patent Application, Publication Number WO 94/01420.

The compositions of the invention are prepared according to conventional practice, such as described in the above mentioned patent applications The dosages of the antagonists may be determined according to conventional methodology such as those described in the above mentioned patent applications.

Lee et al (Nature, 1995, 379, 632-635) have shown that there are a number of forms of the leptin receptor. If the islet receptor(s) differs in nature or proportions to that in other tissues, an especially useful antagonist would be one that antagonizes the action of leptin at the islet and/or skeletal muscle receptor, but does not antagonize other leptin receptors (e.g. in the hypothalamus) and thereby exacerbate insulin resistance.

Mutations in the mouse ob gene (J. Hered. 41, 317-318)1950)) and the db gene (Science 153, 1127-1128 (1966)) result in obesity and non-insulin dependent diabetes. The ob gene product, leptin, is expressed exclusively in adipose tissue (Nature (Lond.), 372, 425-432 (1994)) and it has been demonstrated that daily injections of recombinant leptin inhibits food intake and reduces body weight and fat-mass in ob/ob mice (Science 269, 543-546 (1995); Science 269, 540-543 (1995); Science 269, 546-549 (1995)). Such treatment also results in a reduction in the hyperinsulinaemia (Science 269, 540-543 (1995)). Recently, the gene that codes for the receptor for the ob-gene has been identified (Cell 83, 1263-1271 (1995)). The leptin receptor has several alternatively spliced variants. One of these spliced variants is expressed at a high level in hypothalamus, and is believed to be the functional receptor in the regulation of energy balance. It is abnormally spliced in C57B1/KSJ db/db mice (Nature (Lond.) 379, 622-635 (1996); Cell 84, 491-495 (1996); Science 271, 994-996 (1996)) so that the cytoplasmic region is missing, leading to defective signal transduction. We now report that, in addition to hypothalamus, the predicted functional leptin receptor ob-Rb, is expressed in several tissues, including pancreatic islets, in ob/ob mice. Furthermore, recombinant leptin inhibits directly insulin release from both isolated islets and the perfused pancreas of the ob/ob mouse, and it inhibits basal and insulin-stimulated glycogen synthesis in isolated soleus muscle of ob/ob mice, demonstrating that leptin has both central and peripheral actions.

The diabetic (db) gene product in mice has been identified as the receptor for leptin. At least six alternatively spliced forms of the leptin message have been identified (Nature (Lond.) 379, 622-635 (1996)). One of these, Ob-Rb, has an extensive intracellular region containing a Box 2 sequence motif, which is required for the binding of JAK protein kinases and is believed to encode the functional receptor. Short antisense oligodeoxynucleotide probes (Trayhurn et al, Biochem. Soc. Trans. Vol 23 page 4945, 1995) 30-34 mers, were designed to hybridise with different domains in the mouse leptin receptor mRNA sequence and used to detect either expression of all transcripts of the leptin-receptor or to detect solely the leptin receptor long-form, Ob-Rb. Each oligonucleotide probe sequence was unique to the leptin receptor and had no significant homology to any other known sequence.

Expression in lean +/+mice of total leptin receptor mRNA was detected in hypothalamus, kidneys, lung, liver, whole pancreas, brain, soleus muscle and spleen but not in white adipose tissue, pituitary or heart. Total leptin receptor mRNA, standardised to poly(A) mRNA levels was over-expressed in ob/ob mice relative to the lean littermates. However, there were tissue specific differences in overexpression. Thus, hypothalamus which shows the highest level of expression in lean mice was only 2-fold over-expressed in ob/ob mice. In kidney, total leptin mRNA was over-expressed 4-fold but in some other tissues that had low expression of total leptin mRNA in lean mice, total leptin mRNA was over-expressed by up to 10-fold in ob/ob mice.

Northern blot analysis of hypothalamus probed with a 34-mer corresponding to bases 3329-3363, which are part of the intracellular loop present only in the long-form of this leptin receptor Ob-Rb, gave a single sharp band. This contrasted with the multiple bands when the blot was probed with the 33-mer corresponding to bases 1877-1910, which is a sequence common to all the known splice variants of the leptin receptor. Molecular weight markers indicated that the 34-mer hybridised to a mRNA fragment of approximately 3400 b.p. consistent with the putative functional leptin receptor. This was found to be expressed at a high level in hypothalamus, in agreement with the recent studies of Lee et al (Nature (Lond.) 379, 622-635 (1996)). Furthermore, the hypothalami from ob/ob mice showed 2-fold over-expression relative to the +/+mice. The Ob-Rb leptin receptor is also present (and over-expressed relative to lean +/+mice) in liver, kidney and lung but not in whole brain, heart, soleus muscle or pituitary. The finding of relative over-expression of the Ob-Rb leptin in ob/ob mice relative to lean +/+mice is consistent with the findings of several workers (Science 269, 540-543 (1995); Proc. Natl, Acad. Sci., USA 93, 1726-1730 (1996)) that recombinant leptin is more effective in reducing food intake and body weight in the ob/ob mice. It also raises the possibility that leptin regulates the expression of its receptor. This could result in leptin resistant states and could explain the relative lack of activity of recombinant leptin in dietary induced obesity (Science 269, 540-543 (1995)).

Using the slot-blot technique, no expression of the long-form of the leptin receptor was detected in whole pancreata from either lean +/+or ob/ob mice. However, a strong signal was obtained using mRNA from ob/ob mouse pancreatic islets.

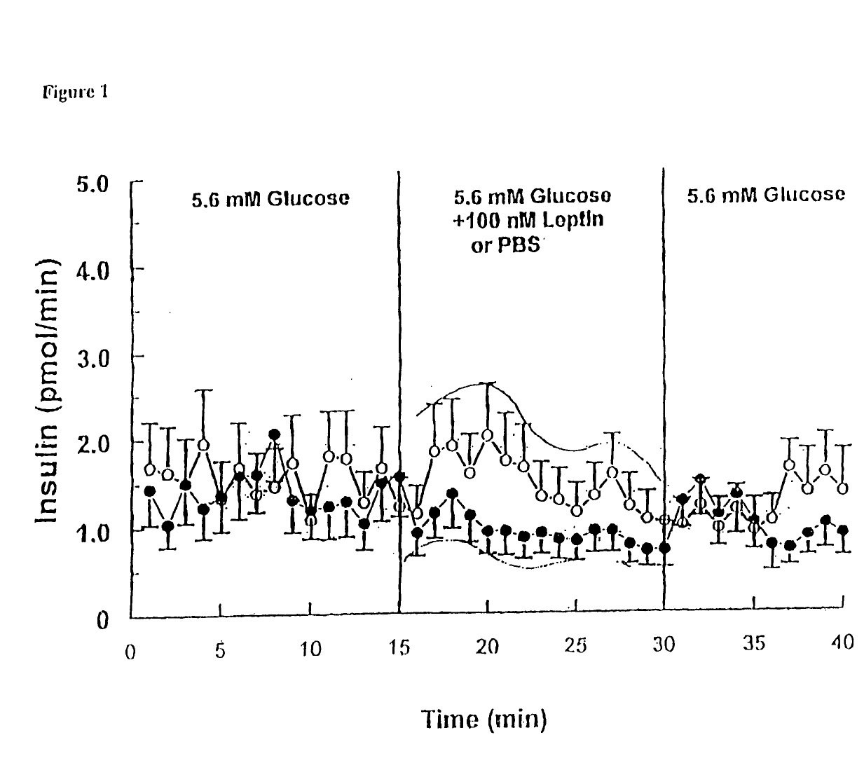

Previous studies have demonstrated that daily intraperitoneal injections of recombinant leptin for 28 days produced a dose-dependent significant reduction in serum insulin and blood glucose in ob/ob mice but not in lean mice (Science 269, 540-543 (1995)). Given the high expression of the long-form of the leptin receptor, which is the putative functional receptor, in pancreatic islets from ob/ob mice, we decided to examine the functional response on insulin secretion using the ob/ob mouse perfused pancreas (FIG. 1, Table 1).

Leptin (100 nM) produced an immediate reduction in the insulin release from the isolated pancreas.

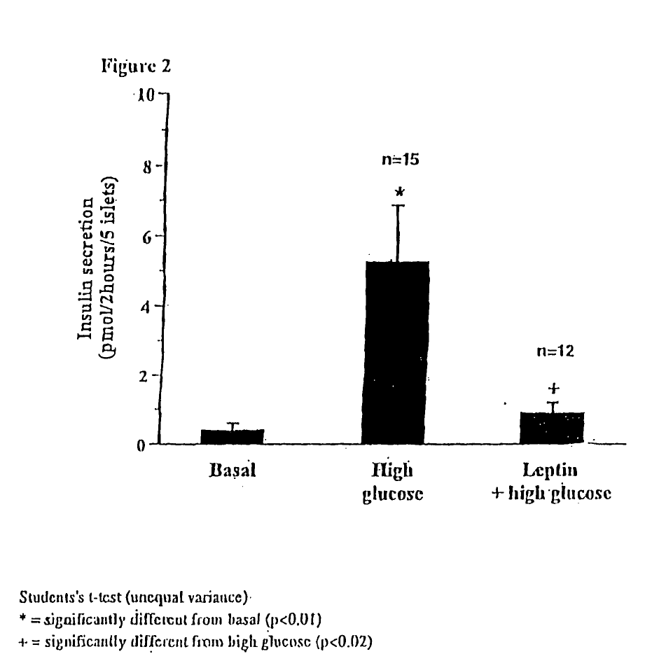

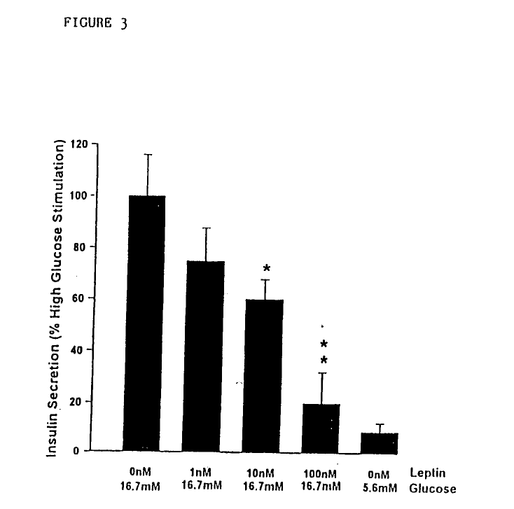

The action of leptin in the perfused pancreas preparation could be either direct on islets or via the release of a further mediator from the vasculature. To clarify this, the effect of leptin on insulin release from ob/ob mouse isolated pancreatic islets was determined. Leptin (100 nM) completely inhibited the stimulatory effect of 16.7 mM glucose on insulin release in islets isolated from overnight-fasted mice (FIG. 2). The inhibitory effect of leptin was dose-related over the range 1-100 nM (FIG. 3). Also leptin (10 nm) inhibited glucose-stimulated insulin secretion by islets from wild-type mice, but leptin (100 nM) had no effect on insulin secretion by islets from ob/ob mice.

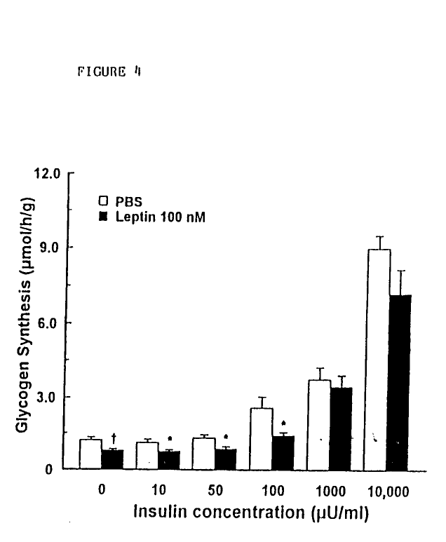

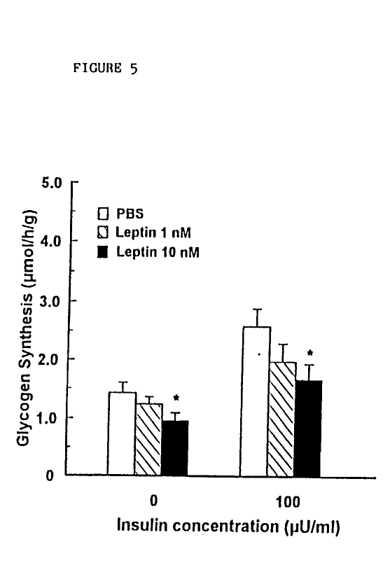

To evaluate the possibility that leptin might directly inhibit glucose uptake and insulin action, [¹⁴C]-glucose incorporation into glycogen was measured in isolated intact soleus muscles, from ob/ob mice, weighing 4-6 mg using the method of Challiss et al. (Biochemical Pharmacology, 1988, 37, 947-950). Recombinant murine leptin at 100 nM inhibited glycogen synthesis in soleus muscle (FIG. 4), with 35% inhibition at basal (P<0.01), and 28%, 30% and 45% at low insulin concentrations (10, 50 and 100 uU/ml respectively, P<0.05). The maximal response to insulin (10,000 uU/ml) was not significantly affected by leptin. The effects of lower concentrations of leptin (1 and 10 nM) were examined in the absence of insulin and in the presence of 100 uU/ml insulin. 10 nM leptin caused significant inhibition of both basal and insulin-stimulated glycogen synthesis (32% and 35% respectively; P<0.05), whereas 1 nM leptin did not have a significant effect (FIG. 5).

Obesity is the commonest nutritional disorder in Western Society and in many developing countries. It is strongly associated with non-insulin dependent diabetes. The basis of this association has largely been assumed to relate to the increase in insulin resistance that occurs with developing adiposity. Insulin resistance might be expected to result in glucose intolerance but it is commonly believed that the development of non-insulin dependent diabetes requires the additional independent development of a pancreatic lesion.

Initial studies using infusion of recombinant leptin to ob/ob mice and lean littermates suggested that the primary action of leptin was to control appetite possibly through a suppression of central NPY release (Nature (Lond.) 337, 530-532 (1995)). However, a more recent study which included a group of ob/ob mice that consumed the same amount of food as leptin infused mice, demonstrated that leptin had significant metabolic actions (Proc. Natl, Acad. Sci., USA 93, 1726-1730 (1996)). The infusion of leptin to ob/ob mice resulted in a significantly lower body weight and fat pad weight relative to pair-fed mice. However, the most dramatic difference between the mice infused with leptin and the pair-fed animals was in the serum insulin concentration. Pair-feeding reduced the insulin concentration from 30.6±6.2 mg/ml to 14.2±4.2 mg/ml. In the mice infused with leptin, the insulin concentration 0.09±0.08 ng/ml was not significantly different from lean animals (Proc. Natl, Acad. Sci., USA 93, 1726-1730 (1996)). The present study demonstrates that the leptin receptor spliced variant that encodes the functional receptor is present in pancreatic islets of ob/ob mice, and that leptin will directly inhibit basal insulin secretion in the perfused pancreas and glucose stimulated insulin release by isolated islets of the ob/ob mice. These data suggest for the first time that leptin over-production following excess adiposity may directly modify insulin secretion and could be involved in the development of the diabetic syndrome. These data further suggest that leptin overproduction associated with obesity may be one of a number of factors responsible for inducing insulin resistance in obesity.

Accordingly, in a further particular aspect the present invention provides the use of an antagonist of leptin for the treatment of insulin resistance, especially that associated with obesity.

Further provided is the use of an antagonist of leptin for the maunfacturemanufacture of a medicament for the treatment of insulin resistance, especially that associated with obesity.

Also provided is a method for the treatment of of insulin resistance, especially that associated with obesity, in a human or non-human mammal, which method comprises the administration to human or non-human mammal in need of such treatment, an effective, pharmaceutically acceptable, non-toxic amount of an antagonist of leptin.

A further particular pharmaceutical composition of the invention is therefore a pharmaceutical composition useful for the treatment of of insulin resistance, especially that associated with obesity.

In addition to pancreatic islets, we have also detected the long-form of the leptin receptor in liver, kidney and lung. The functional effects of leptin in these tissues are at present unknown. However, Levin et al (Proc. Natl, Acad. Sci., USA 93, 1726-1730 (1996)) noted that hepatic glycogen content of liver was significantly reduced in leptin-infused ob/ob mice but not in pair-fed animals. Together with the current data these findings are suggestive that leptin might directly affect hepatic glycogen metabolism.

In summary, the present results present both molecular biology and functional evidence for leptin having widespread peripheral metabolic activity as well as a central action on food intake.

The disclosures of the above mentioned references including patent applicatonsapplications GB2292382 and WO 94/01420 are incorporated herein by reference.

The following Figures and Table illustrate the invention but do not limit it in any way.

TABLE 1

| | Control | Leptin |
|---|---|---|
| AUC 0-15 min | 0.208 ± 0.022 (4) | 0.190 ± 0.028 (5) |
| AUC 16-30 min | 0.196 ± 0.019 (4) | 0.128 ± 0.019 (5)* |
| % change | 96.5 ± 11.3 (4) | 69.2 ± 0.45 (5)* |

0-15 min: Stabilization period
16-30 min: treated period (PBS or Leptin)
AUC: area under the curve
% Change: the means of 16-30 min vs the means of 0-15 min
Results are means ± SEM (n = 4.5).
*P < 0.05 (un-paired test)

1. The use of an antagonist of leptin for the manufacture of a medicament for the treatment of disorders resulting from deficiencies in insulin secretion and of hyperglycaemia.

2. A use according to claim 1, for the treatment of non-insulin-dependent diabetes (NIDDM).

3. The use of an antagonist of leptin for the manufacture of a medicament for the treatment of insulin resistance.

4. A use according to claim 3, for the treatment of insulin resistance associated with obesity.

5. A method for the treatment of disorders resulting from deficiencies in insulin secretion and of hyperglycaemia, in a human or non-human mammal, which method comprises the administration to human or non-human mammal in need of such treatment, an effective, pharmaceutically acceptable, non-toxic amount of an antagonist of leptin.

6. A method for the treatment of insulin resistance, which method comprises the administration to human or non-human mammal in need of such treatment, an effective, pharmaceutically acceptable, non-toxic amount of an antagonist of leptin.

7. A pharmaceutical composition for the treatment of disorders resulting from deficiencies in insulin secretion and of hyperglycaemia, comprising an antagonist of leptin, or a pharmaceutically acceptable derivative thereof, and a pharmaceutically acceptable carrier.

8. A pharmaceutical composition for the treatment of insulin resistance, comprising an antagonist of leptin, or a pharmaceutically acceptable derivative thereof, and a pharmaceutically acceptable carrier.

9. A use according to any one of claims 1 to 8, wherein the antagonist of leptin is a small organic molecule antagonist.
